(19) United States
(12) Patent Application Publication
(10) Pub. No.: US 2021/0060117 A1
(43) Pub. Date: Mar. 4, 2021

(54) ZINC ACTIVATED THYMULIN AND METHODS OF PREPARATION AND ADMINISTRATION

(71) Applicant: CYTOLYF THERAPEUTICS LLC, GARLAND, TX (US)

(72) Inventors: ANAND S. PRASAD, GARLAND, TX (US); ROY A. VARGHESE, GARLAND, TX (US); PHILIP C. BIRD, GARLAND, TX (US); CHARLES CLAYTON, GARLAND, TX (US)

(73) Assignee: CYTOLYF THERAPEUTICS LLC, GARLAND, TX (US)

(21) Appl. No.: 16/993,702

(22) Filed: Aug. 14, 2020

Related U.S. Application Data
(60) Provisional application No. 62/886,995, filed on Aug. 15, 2019.

(51) Int. Cl. A61K 38/08; A61K 33/30; C12N 15/86; G01N 33/50; G01N 33/543; G01N 33/52

(52) U.S. Cl. CPC A61K 38/08; A61K 33/30; C12N 15/86; A61K 9/0053; G01N 33/543; G01N 33/52; G01N 33/5008

(57) ABSTRACT

Embodiments of the invention generally fall into the category of activated thymulin synthesis and applications thereof. Embodiments, delivered orally or parenterally, are used to treat malignancies and immune system dysfunctions by activating cytotoxic T cells, increasing the generation of T helper 1 cells and/or boosting the production of interleukin 2.

### APPLICATION CROSS-REFERENCE

The instant Application relates to U.S. Provisional Patent Application Ser. No. 62/886,995 filed on 15 Aug. 2019, the entirety of which is incorporated by reference.

### SUBMISSION OF SEQUENCE LISTING ON ASCII TEXT FILE

The instant application contains a Sequence Listing which has been submitted electronically in ASCII format and is hereby incorporated by reference in its entirety. Said ASCII file, created on 2 Oct. 2020, is named 1188-0001-1-CytoLyf-SEQ.txt and is 477 bytes in size. The content of the ASCII text file incorporated herein by reference is a computer readable form (CRF) of one sequence:
SEQ. 01 Is the unmodified nonapeptide sequence for thymulin.

### BACKGROUND

### Technical Field

Embodiments of the invention generally fall into the category of activated thymulin synthesis and applications thereof. Embodiments, delivered orally or parenterally, are used to treat malignancies and immune system dysfunctions by activating cytotoxic T cells, increasing the generation of T helper 1 cells and/or boosting the production of interleukin 2.

### Discussion of Art

The thymus plays a key role in mammalian immune systems. It is the primary site for the production of immunocompetent T-lymphocytes. Thymopoietic hormones are responsible for T-lymphocyte marker differentiation, expression and function. A thymic hormone, thymulin is a naturally occurring substance in mammals that can also be synthetically made. Human thymulin has the same amino acid sequence as porcine and bovine thymulin. Synthetic versions of thymulin with the same functionality as biological thymulin were produced using the amino acid sequence of human thymulin (initial syntheses were carried out with solid phase synthesis and classical solution methodologies).

Initially dubbed Thymic Serum Factor (“facteur thymique serique,” or FTS), thymulin is a nonapeptidic hormone that induces expression of several T cell markers, promotes T cell functions (including allogeneic cytotoxicity, suppressor functions and IL-2 production) and acts on both the early and late stages of T lymphocyte differentiation. There are two kinds of thymulin, one inactive and the second active. Thymulin is inactive when deprived of zinc and active when it contains zinc. Thus thymulin is biologically active only when zinc is present. Zinc is an essential trace element for human health particularly in its three major roles: catalytic, structural and regulatory. Zinc in biological organisms is redox-inert; it has one valence state: Zn⁺². One known thymulin:zinc ratio required for biologically active thymulin is 1:1. Zinc is bound to thymulin in a 1:1 stoichiometry via the side chains of asparagine and the hydroxyl groups of the two serines. Thymulin activity, in vitro and in vivo, in both animals and humans, is dependent on plasma zinc concentrations such that marginal changes in zinc intake or availability affect thymulin activity. Evidence gathered thus far suggests that thymulin incorporates zinc before being secreted by thymic epithelial cells.

Due to a requirement for the presence of biologically active thymulin in the differentiation and maturation processes of T-cells, biologically active thymulin is has potential for therapeutic applications. Thymulin is further known to be non-toxic even at relatively high doses. Biologically active thymulin is shown to induce the differentiation of T-cells and enhance several functions of the various T-cell subsets in normal or partially thymus-deficient recipients. Thymulin is detectable in the serum of zinc-deficient patients but it is not active. The binding of zinc to the peptide results in a conformational change that produces the active form of thymulin. FIG. 01 illustrates a schematic diagram of a zinc-thymulin binding structure.

Low levels of thymulin activity may be seen in other conditions such as autoimmune and other chronic diseases. Studies show that in zinc-deficient subjects, the available circulating plasma zinc was unable to generate active thymulin. Thus, an optimal level of serum zinc is required to form active thymulin. It is believed that administration of active thymulin and zinc may have a very useful role in a few immuno-deficient human subjects.

In the peripheral and/or the central nervous system there is evidence that thymulin is capable of modulating pro-inflammatory cytokines in disease suggesting potential for use as an anti-inflammatory. Thymulin is also known to be active on the pituitary gland.

Further, clinical studies suggest that zinc plasma deficiency is present in certain oncological settings. In particular, a zinc plasma deficiency was observed in acute lymphoblastic leukemia patients at the onset and during relapse. Such a deficiency is known to cause a decrease in the activity of thymulin despite nearly normal production by the thymus. The existence of positive correlations between zinc or active thymulin and peripheral immunological parameters (phytohemogglutinin [PHA] and concanavalin A [ConA]) at various stages of the disease suggests a link between derangement of peripheral immune function, thymic hormone activity, and zinc failure.

Thus, studies have shown: zinc is necessary for the biological activity of thymulin; that biologically active thymulin promotes T lymphocyte maturation, IL-2 production and cytotoxicity; and, that immuno-compromised subjects have low levels of biologically active thymulin. Yet, despite the evidence indicating the benefits of biologically active thymulin there has been very little progress in harnessing the therapeutic potential of biologically active thymulin. In particular, there is an inability to systematically generate, customize, measure, monitor and optimize the dosage of biologically active thymulin. Thus, there is a need for methods of preparing and administering active thymulin.

### BRIEF SUMMARY OF THE INVENTION

An embodiment of the invention is a pharmaceutical composition comprising thymulin, zinc, and one or more pharmaceutically acceptable excipients.

In some embodiments, a pharmaceutical composition of the present disclosure comprises synthetically generated zinc-coupled thymulin molecules. In some embodiments, a pharmaceutical composition of the present disclosure comprises synthetically generated thymulin molecules that are then coupled with zinc ex vivo and in vitro.

In another aspect, the present disclosure provides a method for treating, reducing the severity of, reducing the incidence of, delaying the onset of, preventing a relapse to or reducing pathogenesis of a chronic condition associated with an immune system dysfunction or deficiency comprising administering, to a subject in need thereof, a pharmaceutical composition disclosed herein.

In some embodiments, a pharmaceutical composition of the present disclosure comprises synthetically generated zinc-coupled thymulin molecules. In some embodiments, a pharmaceutical composition of the present disclosure comprises synthetically generated thymulin molecules that are then coupled with zinc ex vivo and in vitro.

In some embodiments, a pharmaceutical composition of the present disclosure comprises biological thymulin molecules extracted from human plasma that are then coupled with zinc ex vivo and in vitro.

In some embodiments, a pharmaceutical composition of the present disclosure comprises biological thymulin molecules extracted from porcine plasma that are then coupled with zinc ex vivo and in vitro.

In some embodiments, a pharmaceutical composition of the present disclosure comprises biological thymulin molecules extracted from bovine plasma that are then coupled with zinc ex vivo and in vitro.

In some embodiments, a pharmaceutical composition of the present disclosure comprises zinc ions that are then administered to subjects to generate active thymulin.

In some embodiments, a pharmaceutical composition of the present disclosure is a genetically engineered analog of thymulin that is then administered to subjects to generate active thymulin. In some embodiments the pharmaceutical composition is delivered parenterally to the subject. In some embodiments the pharmaceutical composition is delivered orally to the subject.

In one aspect, the present disclosure provides a method for generating T Helper 1 cells which produce Interleukin 2 which then activates cytotoxic T cells comprising administering, to a subject in need thereof, a pharmaceutical composition disclosed herein.

In another aspect, the present disclosure provides a pharmaceutical composition comprising: the recombinant adenoviral vector, RAd-metFTS (methionine-FTS), expressing the synthetic DNA sequence encoding met-FTS; a biologically active analog of Serum Thymic Factor (FTS) (in this framework, metFTS is constructed and cloned in different adenoviral vectors); and, pharmaceutically acceptable excipients.

It is further an object of the invention to create biologically active thymulin compositions ex vivo and in vitro by adding zinc to synthetic or biological forms of thymulin.

It is still another object of the invention to confirm the biological activity of thymulin in pharmaceutical compositions ex vivo and in vitro using one or more measurement technologies including those capable of molecular level analysis.

In yet another aspect, it is an object of the invention to deliver the compositions disclosed herein to subject via oral and parenteral administration.

Another object of the invention is to monitor the therapeutic efficacy of the compositions disclosed herein in subjects with biomarkers such as T cell activity on a continuous basis so as to modulate and optimize dosage and delivery.

In another aspect, an object of the invention is to treat malignancies and immune system dysfunctions by activating cytotoxic T Cells that then contribute to the mitigation of the dysfunction or malignancy.

### BRIEF DESCRIPTION OF THE DRAWINGS

FIG. 01 is an example diagram of the zinc-thymulin binding structure.

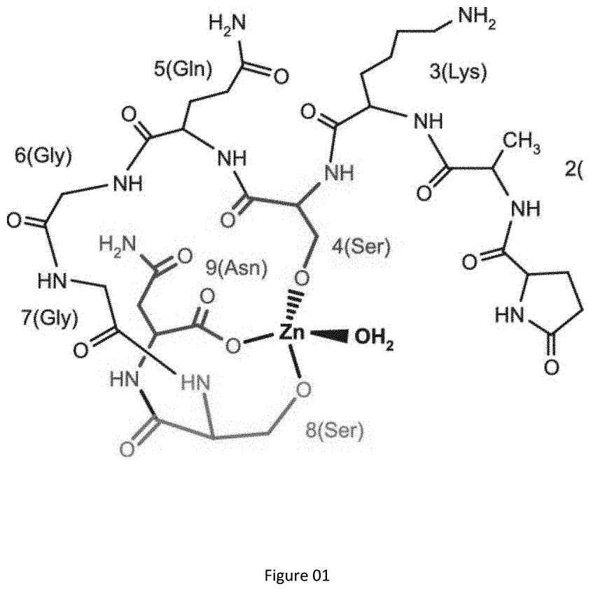

### DETAILED DESCRIPTION OF THE INVENTION

Unless otherwise defined, all technical and scientific terms used herein possess the meaning commonly understood by the skilled artisan. In the case of inconsistencies, the present disclosure, including definitions, controls.

As used above, and throughout the description, the following terms, unless otherwise indicated, shall be understood to have the following meanings:

As used herein, “a” (or “an”), “one or more,” and “at least one” can be used interchangeably and refer to one or more of an entity. For example, at least one excipient refers to one or more excipients.

As used herein, “a pharmaceutical composition” refers to “one or more pharmaceutical compositions.”

As used herein, “about” means within 10%, such as within 5% and further such as within 2.5%, of a given value or range. When the term “about” is used in conjunction with a numerical range, it modifies that range by extending the boundaries above and below the numerical values set forth. The term “about” may also indicate reasonable tolerances and variations reflected in preparations and compositions under manufacturing processes.

As used herein, an “active ingredient” is an ingredient in a pharmaceutical composition that is biologically active (i.e., alters a chemical or physiological function of a cell, tissue, organ, or organism). In the case of thymulin, “active thymulin” is thymulin with associated zinc sufficient to render the thymulin capable of creating a biologic effect regardless of the source of zinc and/or the presence of additional ions, compounds, enzymes, peptides, small molecules, etc.

As used herein, a “pharmaceutically acceptable excipient” is a functional or non-functional ingredient in a pharmaceutical composition other than the active ingredient(s) useful in preparing said pharmaceutical composition. A “pharmaceutically acceptable excipient” is generally safe and acceptable for mammalian pharmaceutical use.

As used herein, a “disintegrant” is a pharmaceutically acceptable excipient that hydrates a pharmaceutical composition and facilitates the disintegration or breakup of a pharmaceutical composition (e.g., a tablet).

As used herein, a “diluent” or “filler” is an excipient that dilutes the active ingredient(s) and adds bulkiness to a pharmaceutical composition. For example, a diluent or filler may stabilize the active ingredient(s) or facilitate compression.

As used herein, a “surfactant” is an excipient that imparts pharmaceutical compositions with enhanced solubility and/or wettability.

As used herein, a “binder” is a pharmaceutically acceptable excipient that imparts a pharmaceutical composition with cohesive qualities or tensile strength (e.g., hardness).

As used herein, a “glidant” is a pharmaceutically acceptable excipient that imparts a pharmaceutical composition with enhanced flow properties, thereby preventing, reducing, or inhibiting adhesion or friction during processing.

As used herein, a “lubricant” is a pharmaceutically acceptable excipient that imparts improved compaction and ejection properties to a pharmaceutical composition by preventing the active ingredient(s) from clumping together and sticking to manufacturing equipment.

As used herein, “encapsulation machinery” refers to any machine or piece of equipment that may be used to facilitate capsule filling. Encapsulation machinery may be automatic, semiautomatic, or manual.

As used herein, “tableting machinery” refers to any machine or piece of equipment that may be used to facilitate tablet production. Tableting machinery may be automatic, semiautomatic, or manual.

As used herein, “% w/w” refers to the weight percentage of an ingredient in a pharmaceutical composition. For example, 5% w/w means that the weight of an ingredient is 5% of the total weight of the pharmaceutical composition. The total weight of the pharmaceutical composition includes the weight of the ingredient.

As used herein, “% w/v” refers to the weight/volume percentage of an ingredient in a pharmaceutical composition. For example, 5% w/v means that the weight of an ingredient is 5% of the total volume of the pharmaceutical composition. The total weight of the pharmaceutical composition includes the weight of the ingredient.

As used herein, “daily dosage” refers to the total quantity of an active ingredient consumed in the form of a pharmaceutical composition. As used herein, the daily dosage of an active ingredient does not include active ingredient consumed via normal eating behaviors (i.e., dietary sources of the active ingredient).

As used herein, “immune system dysfunctions” refers to any weakening of the body's immune responses to pathogens, viruses, bacteria, foreign bodies, and mutated cells

As used herein, “malignancy” refers to “diseases in which abnormal cells divide without control and can invade nearby tissues. Malignant cells can also spread to other parts of the body through the blood and lymph systems. There are several main types of malignancy. Carcinoma is a malignancy that begins in the skin or in tissues that line or cover internal organs. Sarcoma is a malignancy that begins in bone, cartilage, fat, muscle, blood vessels, or other connective or supportive tissue. Leukemia is a malignancy that starts in blood-forming tissue, such as the bone marrow, and causes large numbers of abnormal blood cells to be produced and enter the blood. Lymphoma and multiple myeloma are malignancies that begin in the cells of the immune system. Central nervous system cancers are malignancies that begin in the tissues of the brain and spinal cord.

As used herein, “T-helper 1 cells” refer to a specialized population of T cells. They are important for immune responses against bacteria and viruses that invade cells. They are characterized by their production of a pro-inflammatory molecule known as interferon-gamma. Adaptive immune responses are tailored to different types of pathogens through differentiation of naive CD4 T cells into functionally distinct subsets of effector T cells (T helper 1 (TH1), TH2, and TH17) defined by expression of the key transcription factors T-bet, GATA3, and RORγt, respectively.

As used herein, “Interleukin-2” refers to one of a group of related proteins made by leukocytes (white blood cells) and other cells in the body. Interleukin-2 is made by a type of T lymphocyte. It increases the growth and activity of other T lymphocytes and B lymphocytes and affects the development of the immune system.

As used herein, “Cytotoxic T cell” refers to a type of immune cell that can kill certain cells, including foreign cells, cancer cells, and cells infected with a virus. Cytotoxic T cells can be separated from other blood cells, grown in the laboratory, and then given to a patient to kill cancer cells. A cytotoxic T cell is a type of white blood cell and a type of lymphocyte. Also called cytotoxic T lymphocyte and killer T cell.

Other embodiments of the current invention provide for pharmaceutical compositions comprising the addition of zinc to thymulin, resulting in the subsequent activation of the thymulin as described. These compositions may further comprise a pharmaceutically acceptable excipient, carrier, or diluent and do not contain any biologically harmful substances. The pharmaceutical compositions of the present invention may be formulated by one having ordinary skill in the art. Suitable pharmaceutical formulations are described in Remington's Pharmaceutical Sciences which is a standard reference text in the field which is herein incorporated by reference.

The pharmaceutical compositions may further comprise coloring or stabilizing agents, osmotic agents, antibacterial agents, or any other substances as long as such substances do not interfere with the function of the composition. The pharmaceutical compositions of the instant invention, can, for example, be formulated as a solution, suspension, or emulsion in association with a pharmaceutically acceptable parenteral vehicle. Examples of such vehicles are water, saline, Ringer's solution, dextrose solution, and 5% human albumen. Liposomes may also be used. The vehicle may contain additives that maintain isotonicity (e.g., sodium chloride or mannitol) and chemical stability (e.g., buffers and preservatives). It should be appreciated that endotoxin contamination should be kept at a safe level, for example, less than 0.5 ng mg⁻¹ protein. Moreover, for human administration, preparations should meet sterility, pyrogenicity, general safety and purity standards as required by the United States Food and Drug Administration Office of Biological Standards. The formulations may be sterilized by commonly used techniques such as filtration.

The phrase “pharmaceutically acceptable” refers to substances and compositions which do not produce an adverse, allergic, or otherwise untoward reaction when administered to an animal, or a human, as appropriate. A substance which caused or produced any of these adverse effects would be classified as “biologically harmful’ within the scope of the present invention. Pharmaceutically acceptable substances and compositions include, but are not limited to solvents, dispersion media, coatings, antibacterial and antifungal agents, isotonic and absorption delaying agents. Except where incompatible with the invention the use of any conventional ingredient is contemplated. Furthermore, supplementary active ingredients which serve some other pharmacologically expedient purpose can also be incorporated into the instant compositions without departing from the broader scope of the instant invention.

A treatment dosage is used as commonly understood in the art as one calculated to immediately or gradually (the terms “immediately” or “gradually” understood to be qualitative and relative, not quantitative) produce a relatively fast treatment effect in a patient. A maintenance dosage, as commonly understood, is one calculated to sustain a treatment effect in a patient. An initial treatment dosage may be decreased to a maintenance dosage. In similar fashion, maintenance dosages may be increased or decreased as demanded by one or more physiological measurements or in conjunction with additional treatment protocols.

The effective dose and method of administration of a particular embodiment of the instant invention may vary based on the individual patient and stage of any present diseases (e.g., cancers, thyroid disorders, immune disorders, and/or other co-morbidities), as well as other factors known to those of skill in the art. Therapeutic efficacy and toxicity of such embodiments can be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies may be used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The exact dosage for any embodiment of the present invention may be chosen by an individual physician in view of a patient to be treated. Dosage and administration are adjusted to provide sufficient levels of embodiments of the instant invention to maintain the desired effect. Additional factors that may be taken into account include the severity of any disease state, age, weight, and gender of the patient; diet, time and frequency of the administration, drug combination(s), reaction sensitivities, and tolerance/response to therapy. Short acting pharmaceutical compositions may be administered daily whereas long acting pharmaceutical compositions may be administered every 2, 3 to 4 days, every week, or once every two weeks or more. Depending on half-life and clearance rate of the particular formulation, the pharmaceutical compositions of the instant invention may be administered once, twice, three, four, five, six, seven, eight, nine, ten or more times per day.

The molecular formula for thymulin is C₃₃H₅₄N₁₂O₁₅. Thymulin, a nonapeptidic hormone with a base sequence illustrated by SEQ. 01, has the following possible peptide sequences:

A. Glu-Ala-Lys-Ser-Gln-Gly-Gly-Ser-Asn-OH
B. H-Pyr-Glu-Ala-Lys-Ser-Gln-Gly-Gly-SerAsn-OH
(when pyroglutamic acid is added)
C. Pyr-Glu-Ala-Lys-Ser-GIn-Gly-Gly-Ser-Asn
D. H-Pyr-Ala-Lys-Ser-Gln-Gly-Gly-Ser-Asn-OH
E. Pyr-Ala-Lys-Ser-GIn-Gly-Gly-Ser-Asn-OH

Also, of note, thymulin is not to be confused with other thymic hormones such as thymosin or thymosin-based products such as thymostimulin which are intended to stimulate T cell proliferation and differentiation but have no bearing on the pharmacology of thymulin.

Simple zinc supplementation in and of itself is not sufficient for activating thymulin. There are inhibitors to thymulin binding with zinc when a subject in need receives zinc supplementation. For instance, α2-macroglobulin competes with thymulin for zinc binding and this competition is thought to be responsible for active thymulin deficits. Moreover, even with supplementation many obstacles stand in the way of zinc absorption ranging from the kind of zinc salt administered, the time of administration, the concurrent consumption of zinc antagonistic minerals, etc. The present disclosure overcomes all these challenges through the novel method of delivering pre-activated thymulin.

Another key obstacle in harnessing the benefits of biologically active thymulin is the absence of any practical capability for detecting and deploying it. The biological activity of thymulin is dependent upon it binding one molecule of zinc. An assay known as the rosette inhibition assay by Dardenne & Bach, besides being cumbersome, represents the only method available to evaluate the biologically active form of the hormone, as immunoassays cannot discriminate between active thymulin (or Zn-Facteur Thymique Sérique (Zn-FTS)) and the inactive form (i.e. not containing Zn, or FTS). The present disclosure overcomes this challenge with its novel method of delivering already activated thymulin. Further, the present disclosure also includes the use of newer sophisticated technologies designed to detect and thereby optimize active thymulin.

Finally, the value of the present disclosure becomes apparent given the health-detrimental decrease of active thymulin over time as a result of one or more thymulin-inhibiting factors. Without being bound to any particular theory or interpretation, although the influence of a putative inhibitory factor on thymulin age-related fluctuations can be hypothesized, the mechanism of this inhibition (enzymatic degradation, binding to a carrier protein, inhibitory factors acting at the level of target cells) remains to be elucidated. In previous studies the levels of a thymulin inhibitory factor appear to increase after age 30 years, reaching very high levels in subjects aged >70 years. This is yet another barrier eliminated by the present disclosure with its focus on not only providing active thymulin but on optimizing its activity through its regime of monitoring, measurement and customized delivery.

In some embodiments, the addition of zinc to thymulin and the subsequent activation of thymulin described herein involves a solution with neutral or slightly alkaline pH (7-8.5) at room temperature or 37° C. Activation will take approximately five minutes. The zinc salt is added at 1-500 ppm in distilled water. Synthetic thymulin salts (trifluoroacetate) can be added to a solvent at a ratio of 1 mg to 1 ml. The zinc and thymulin solutions with can be combined in different molar ratios with a 1:1 equimolar concentration being the preferred combination.

Lyophilized synthetic thymulin is normally reconstituted with 0.1% acetic acid, a standard solvent for salts. The thymulin present in biologically extracted serum is quantitatively determined with an ELISA kit. In a typical such assay kit, the test principle is based on a competition between the antigen in the sample and biotinylated thymulin as a tracer for the binding sites of anti-Thymulin antibodies coated on the wells of the microplate. A peroxidase-conjugated steptavidin is used for detection and quantification, and tetramethylbenzidine (TMB) as a peroxidase substrate. The enzymatic reaction is terminated by an acidic stop solution. A dose response curve of the absorbance unit (optical density, OD at 450 nm) vs. concentration is generated, using the values obtained from the standards. Thymulin present in the patient samples is determined directly from this curve.

In one aspect, the present disclosure provides a pharmaceutical composition comprising thymulin, zinc and one or more pharmaceutically acceptable excipients.

In some embodiments, the zinc is selected from zinc acetate, zinc chloride, zinc sulfate, zinc monomethionine, zinc picolinate, zinc gluconate, zinc aspartate, zinc citrate, zinc orotate, zinc glycinate, zinc oxide, and mixtures thereof. In some embodiments, the zinc is zinc acetate. In some embodiments, the zinc is zinc chloride. In some embodiments, the zinc is zinc sulfate. In some embodiments, the zinc is zinc monomethionine. In some embodiments, the zinc is zinc picolinate. In some embodiments, the zinc is zinc gluconate. In some embodiments, the zinc is zinc aspartate. In some embodiments, the zinc is zinc citrate. In some embodiments, the zinc is zinc orotate. In some embodiments, the zinc is zinc glycinate. In some embodiments, the zinc is zinc oxide.

In some embodiments, the thymulin is selected from thymulin sequenced and synthesized using the amino acid sequence Glu-Ala-Lys-Ser-Gln-Gly-Gly-SerAsn-OH. In some embodiments, the thymulin is selected from thymulin sequenced and synthesized using the amino acid sequence H-Pyr-Glu-Ala-Lys-Ser-Gln-Gly-Gly-SerAsn-OH. In some embodiments, the thymulin is selected from thymulin sequenced and synthesized using the amino acid sequence Pyro-Glu-Ala-Lys-Ser-GIn-GlyGly-Ser-Asn. In some embodiments, the thymulin is selected from thymulin sequenced and synthesized using the amino acid sequence H-Pyr-Ala-Lys-Ser-Gln-Gly-Gly-Ser-Asn-OH. In some embodiments, the thymulin is selected from thymulin sequenced and synthesized using the amino acid sequence Pyr-Ala-Lys-Ser-GIn-Gly-Gly-Ser-Asn-OH.

In some embodiments, the thymulin is produced by human thymic epithelial cells and biologically extracted from human plasma using standard procedures. In some embodiments, the thymulin is biologically extracted from porcine plasma using standard procedures. In some embodiments, the thymulin is biologically extracted from bovine plasma using standard procedures. In some embodiments, the thymulin is secured from genetically engineered analogs of the thymulin peptide.

In some embodiments, zinc is present in an amount ranging from about 10 mg to about 100 mg. For example, in some embodiments, zinc is present in an amount of about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In some embodiments, zinc is present in an amount of about 35 mg. In some embodiments, zinc is present in an amount of about 40 mg. In some embodiments, zinc is present in an amount of about 45 mg. In some embodiments, zinc is present in an amount of about 50 mg. In some embodiments, zinc is present in an amount of about 55 mg.

In some embodiments, zinc is present in an amount ranging from about 15 micromolar zinc to about 45 micromolar zinc. For example, in some embodiments, zinc is present in an amount of about 15 micromolar zinc, about 20 micromolar zinc, 30 micromolar zinc or about 45 micromolar zinc. In some embodiments, zinc is present in an amount of about 15 micromolar zinc. In some embodiments, zinc is present in an amount of about 20 micromolar zinc. In some embodiments, zinc is present in an amount of about 30 micromolar zinc. In some embodiments, zinc is present in an amount of about 45 micromolar zinc.

In some embodiments, thymulin is present in an amount ranging from about 0.4 mg/kg to 1.5 mg/body mass of the subject. In some embodiments, thymulin is present in an amount of 0.4 mg/kg body mass of the subject. In some embodiments, thymulin is present in an amount of 1.5 mg/kg body mass of the subject.

In some embodiments, thymulin is present in the range of 0.1-100 μg kg⁻¹.

In some embodiments, thymulin is present in the range of 5 mg to 20 mg.

In some embodiments, thymulin is present in an amount ranging from about 10 picograms per milliliter to 4000 picograms per milliliter.

In some embodiments, a pharmaceutical composition of the present disclosure comprises synthetically generated zinc-coupled thymulin molecules. In some embodiments, a pharmaceutical composition of the present disclosure comprises synthetically generated thymulin molecules that are then coupled with zinc ex vivo and in vitro.

In some embodiments, a pharmaceutical composition of the present disclosure comprises biological thymulin molecules extracted from human plasma that are then coupled with zinc ex vivo and in vitro. In some embodiments, a pharmaceutical composition of the present disclosure comprises biological thymulin molecules extracted from porcine plasma that are then coupled with zinc ex vivo and in vitro. In some embodiments, a pharmaceutical composition of the present disclosure comprises biological thymulin molecules extracted from bovine plasma that are then coupled with zinc ex vivo and in vitro. In some embodiments, a pharmaceutical composition of the present disclosure comprises zinc ions that are then administered to subjects to generate active thymulin. In some embodiments, a pharmaceutical composition of the present disclosure is a genetically engineered analog of thymulin that is then administered to subjects to generate active thymulin. In some embodiments the pharmaceutical composition is delivered parenterally to the subject. In some embodiments the pharmaceutical composition is delivered orally to the subject.

In a preferred embodiment, a solution of 15 micromolar zinc is added to an equimolar solution of synthetic thymulin along with pharmaceutically acceptable excipients and then administered parenterally to the subject.

In a preferred embodiment, a solution of 20 micromolar zinc is added to an equimolar solution of synthetic thymulin along with pharmaceutically acceptable excipients and then administered parenterally to the subject.

In a preferred embodiment, a solution of 30 micromolar zinc is added to an equimolar solution of synthetic thymulin along with pharmaceutically acceptable excipients and then administered parenterally to the subject.

In a preferred embodiment, a solution of 45 micromolar zinc is added to an equimolar solution of synthetic thymulin along with pharmaceutically acceptable excipients and then administered parenterally to the subject.

In a preferred embodiment, a solution of 15 micromolar zinc is added to an equimolar solution of natural (human, porcine or bovine) thymulin along with pharmaceutically acceptable excipients and then administered parenterally to the subject.

In a preferred embodiment, a solution of 20 micromolar zinc is added to an equimolar solution of natural (human, porcine or bovine) thymulin along with pharmaceutically acceptable excipients and then administered parenterally to the subject.

In a preferred embodiment, a solution of 30 micromolar zinc is added to an equimolar solution of natural (human, porcine or bovine) thymulin along with pharmaceutically acceptable excipients and then administered parenterally to the subject.

In a preferred embodiment, a solution of 45 micromolar zinc is added to an equimolar solution of natural (human, porcine or bovine) thymulin along with pharmaceutically acceptable excipients and then administered parenterally to the subject.

In a preferred embodiment, a solution of 15 micromolar zinc is added to an equimolar solution of synthetic thymulin along with pharmaceutically acceptable excipients and then administered orally to the subject.

In a preferred embodiment, a solution of 20 micromolar zinc is added to an equimolar solution of synthetic thymulin along with pharmaceutically acceptable excipients and then administered orally to the subject.

In a preferred embodiment, a solution of 30 micromolar zinc is added to an equimolar solution of synthetic thymulin along with pharmaceutically acceptable excipients and then administered orally to the subject.

In a preferred embodiment, a solution of 45 micromolar zinc is added to an equimolar solution of synthetic thymulin along with pharmaceutically acceptable excipients and then administered orally to the subject.

In a preferred embodiment, a solution of 15 micromolar zinc is added to a solution of 15 micromolar synthetic thymulin along with pharmaceutically acceptable excipients and then administered parenterally to the subject.

In a preferred embodiment, a solution of 20 micromolar zinc is added to a solution of 20 micromolar synthetic thymulin along with pharmaceutically acceptable excipients and then administered parenterally to the subject.

In a preferred embodiment, a solution of 30 micromolar zinc is added to a solution of 30 micromolar synthetic thymulin along with pharmaceutically acceptable excipients and then administered parenterally to the subject.

In a preferred embodiment, a solution of 45 micromolar zinc is added to a solution of 45 micromolar synthetic thymulin along with pharmaceutically acceptable excipients and then administered parenterally to the subject.

In some embodiments, Zinc and thymulin are present at a weight/volume percentage ranging from 0.0001%-0.1%.

In some embodiments, Zinc and thymulin are present at a weight/volume percentage ranging from 0.001%-0.05%.

In another aspect, the present disclosure provides a pharmaceutical composition comprising:

about 10 mg to about 200 mg zinc;

about 5 to 20 mg of thymulin; and,

pharmaceutically acceptable excipients.

In another aspect, the present disclosure provides a pharmaceutical composition comprising:

about 15 micromolar to 100 micromolar zinc;

about 0.1 mg/kg to 10 mg/kg body mass thymulin; and,

pharmaceutically acceptable excipients.

In another aspect, the present disclosure provides a pharmaceutical composition comprising:

about 30 mg zinc;

about 0.4 mg/kg of body mass of thymulin; and,

pharmaceutically acceptable excipients.

In another aspect, the present disclosure provides a pharmaceutical composition of claim 1 comprising:

about 15 micromolar zinc;

about 0.4 mg/kg of body mass of thymulin; and,

pharmaceutically acceptable excipients.

In another aspect, the present disclosure provides a pharmaceutical composition comprising:

about 15 to 45 micromolar zinc; and,

pharmaceutically acceptable excipients.

In another aspect, the present disclosure provides a pharmaceutical composition comprising:
the recombinant adenoviral vector, RAd-metFTS (methionine-FTS), expressing the synthetic DNA sequence encoding met-FTS;
a biologically active analog of Serum Thymic Factor (FTS) (in this framework, metFTS is constructed and cloned in different adenoviral vectors); and,
pharmaceutically acceptable excipients.

In some embodiments, the total weight percentage of pharmaceutical excipient(s) in a pharmaceutical composition disclosed herein is up to about 35% w/w. For example, in some embodiments, the total weight percentage of pharmaceutical excipient(s) in a pharmaceutical composition disclosed herein is up to about 35% w/w, up to about 30% w/w, up to about 25% w/w, up to about 20% w/w, up to about 15% w/w, or up to about 10% w/w.

In some embodiments, a pharmaceutical composition disclosed herein is formulated as a liquid to be administered parenterally or orally. In some embodiments, a pharmaceutical composition disclosed herein is formulated as a solid oral dosage form. Non-limiting examples of solid oral dosage forms include tablets, such as a sugar-coated tablet, a film-coated tablet, a sublingual tablet, a buccal tablet, or an orally disintegrating oral tablet, and capsules, such as a soft capsule or microcapsule. Solid and liquid parenteral and oral dosage forms of the present disclosure may be prepared by any known production method generally used in the technical field of pharmaceuticals preparation. The thymulin used in the composition can be isolated from human, porcine or bovine serum or it can be generated by ordinary chemical synthesis involving solid or liquid phase reactions or it can be prepared by a genetic-engineering process and/or a cell-fusion process. In particular embodiments, parenteral and oral dosage forms provided herein may be prepared using conventional methods known to those skilled in the field of pharmaceutical preparation, as described, e.g., in pertinent textbooks. See, e.g., Thymic Hormones and Lymphokines: Basic Chemistry and Clinical Applications (Springer, 1984) by Allan Goldstein, the five-volume Amino Acids, Peptides and Proteins in Organic Chemistry, Building Blocks, Catalysis and Coupling Chemistry (Wiley, 2011) by Andrew Hughes and Chemistry of Peptide Synthesis (CRC Press, 2005) by N. Leo Benoiton. These references are hereby incorporated herein by reference to the extent they disclose suitable, conventional methods known to those skilled in the field of pharmaceutical formulation.

In some embodiments, a pharmaceutical composition of the present disclosure comprises filler, a binder, a disintegrant, a lubricant, a glidant, buffer salts to help improve pH control, preservative excipients, solubilizing excipients such as surfactants, co-solvents and cyclodextrins, stabilization excipients, bulking agents in lyophilized formulations and tonicity agents.

In some embodiments, a pharmaceutical composition of the present disclosure comprises acid-resistant enteric coating, peptidase inhibitors, pH modifiers, polymer micro-/nanoparticles, solid lipid micro-/nanoparticles, microemulsions, liposomes, and combinations of the above.

In some embodiments, a pharmaceutical composition disclosed herein is formulated as one, two, or three solid or liquid dosage forms. In some embodiments, a pharmaceutical composition disclosed herein is formulated as one solid oral dosage form. In some embodiments, a pharmaceutical composition disclosed herein is formulated as two solid oral dosage forms. In some embodiments, a pharmaceutical composition disclosed herein is formulated as three solid oral dosage forms.

In some embodiments, the pharmaceutical compositions disclosed herein are formulated as a powder.

In some embodiments, the pharmaceutical composition disclosed herein may be formulated as a liquid suitable for oral ingestion.

In some embodiments, the pharmaceutical composition disclosed herein may be formulated as a liquid suitable for intravenous or subcutaneous or intramuscular administration.

In some embodiments, the pharmaceutical composition disclosed herein may be orally administered to patients in the form of any pharmaceutically acceptable salt. In some embodiments, the pharmaceutical composition disclosed herein may be administered to patients by a parenteral route.

In some embodiments, the pharmaceutical composition disclosed herein may be administered to patients by intravenous, intramuscular or subcutaneous injections. In some embodiments, the pharmaceutical composition disclosed herein may be administered in the form of a suppository.

In one aspect, the presence of active thymulin in the pharmaceutical composition disclosed herein is monitored and optimized by using X Ray fluorescence (XRF) techniques whereby XRF analyzers delineate the chemical makeup of the composition by monitoring the fluorescent X-rays generated by it when irradiated by an X-ray source subsequent to which the specific elements present in it generate their unique fluorescent X-ray fingerprints. In some embodiments, the X Ray fluorescence method enables detection of the zinc present in thymulin at a subatomic level.

In another embodiment, the presence of active thymulin in the pharmaceutical composition can be detected using the enzyme-linked immunosorbent assay (ELISA) kit that determines the presence of thymulin in the composition within a range of 0.03-16 ng mL⁻¹ In another embodiment, the measurement method is a radioimmunoassay of the composition using an antibody specific for thymulin. In some instances the antibody may be a monoclonal antibody or the antibody from the antiserum of a host animal; and a radiolabeled thymulin analogue as the tracer. In another embodiment, the zinc in the pharmaceutical composition is measured using atomic absorption spectroscopy.

In one aspect, the present disclosure provides a method for generating T Helper 1 cells which produce Interleukin 2 which then activates cytotoxic T cells comprising administering, to a subject in need thereof, a pharmaceutical composition disclosed herein.

In another aspect, the present disclosure provides a method for treating, reducing the severity of, reducing the incidence of, delaying the onset of, preventing a relapse to or reducing pathogenesis of a chronic condition associated with an immune system dysfunction or deficiency comprising administering, to a subject in need thereof, a pharmaceutical composition disclosed herein.

In some embodiments, the chronic condition being treated is cancer including pancreatic cancer, prostate cancer, breast cancer, lung cancer, colon cancer, cervical cancer, ovarian cancer, melanoma, lymphoma and squamous cell carcinoma.

In another aspect, the present disclosure provides a method for treating, reducing the severity of, reducing the incidence of, delaying the onset of, preventing a relapse to or reducing pathogenesis of a persistent infection comprising administering, to a subject in need thereof, a pharmaceutical composition disclosed herein.

In some embodiments, the persistent infection being treated is a viral infection, a bacterial infection, a fungal infection, or a parasitic infection. In some embodiments, the viral infection is an infection with a hepatitis virus, a human immunodeficiency virus (HIV), a human T-lymphotrophic virus (HTLV), a herpes virus, an Epstein-Barr virus, or a human papilloma virus.

In another aspect, the present disclosure provides a method for selecting a treatment for a subject having or being at risk of having a persistent infection or cancer comprising the steps of (a) securing a plasma sample from the subject; (b) measuring the biological activity of thymulin in the sample; and (c) selecting a treatment for the subject diagnosed as having or being at risk of a persistent infection or cancer which could include administering a pharmaceutical composition disclosed herein.

In one aspect, the generation of T Helper 1 cells and other immune cells is measured using standard methods such as the cytotoxic T Lymphocyte (CTL) response assay, the ELISPOT (enzyme-linked immunospot) technique, flow cytometric analyses of intracellular cytokines, an optofluidics method to isolate, culture and assayed individual cells, and other methodologies.

In some embodiments, the daily dose of the pharmaceutical composition disclosed herein ranges from about 10 mg to about 200 mg zinc and about 5 to 20 mg of thymulin and pharmaceutically acceptable excipients. In some embodiments, the daily dose of the pharmaceutical composition disclosed herein ranges from about 15 micromolar to 100 micromolar zinc and 15 micromolar to 100 micromolar thymulin. In some embodiments, the daily dose of the pharmaceutical composition disclosed is 15 micromolar zinc and 15 micromolar thymulin. In some embodiments, the daily dose of the pharmaceutical composition disclosed is 20 micromolar zinc and 20 micromolar thymulin.

In some embodiments, the pharmaceutical composition described herein is administered from one to three times per day. In some embodiments, the pharmaceutical composition described herein is administered one time per day. In some embodiments, the pharmaceutical composition described herein is administered multiple times per day. In some embodiments, the pharmaceutical composition is administered every second day or every third day.

It is anticipated that subjects experiencing chronic infection (including viral infections), as described above, administered active thymulin in accordance with an embodiment of the invention, will have increased serum levels of activated thymulin resulting in at least increased production of T Helper 1 cells, increased production of Interleukin 2, or increased cytotoxic T cell activity the net effect to shorten the duration of and/or eliminate the chronic infection in the subject.

It is further anticipated that subjects experiencing malignancies, as described above, administered active thymulin in accordance with an embodiment of the invention, will have increased serum levels of activated thymulin resulting in at least increased production of T Helper 1 cells, increased production of Interleukin 2, or increased cytotoxic T cell activity the net effect to shorten the duration of and/or eliminate the malignancy in the subject.

Finally, the written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

The pharmacologically active compounds of this invention can be processed in accordance with conventional methods of galenic pharmacy to produce medicinal agents for administration to patients (e.g., mammals including humans).

As used herein the term “sequence” explicitly contemplates DNA, cDNA, RNA and resulting peptide chains encoded thereby in both sense and antisense directions. To know one is to know the others via the standard rules of complementarity and codon encoding as exemplified in standardized DNA, RNA, and amino acid codon tables.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

Since certain changes may be made in the above-described invention, without departing from the spirit and scope of the invention herein involved, it is intended that all of the subject matter of the above description shown in the accompanying drawings shall be interpreted merely as examples illustrating the inventive concept herein and shall not be construed as limiting the invention.

### REFERENCES

The following are hereby incorporated by reference in their entirety for the teachings they contain:
A S Prasad, J F Bach, M Dardenne, et al., “Serum thymulin in human zinc deficiency,” Journal of Clinical Investigation, 1988; 82(4):1202-1210, https://doi.org/10.1172/JCI113717.
Ananda S. Prasad, “Lessons Learned from Experimental Human Model of Zinc Deficiency,” Journal of Immunology Research, Volume Jan. 9, 2020, Volume 2020, https://doi.org/10.1155/2020/9207279
Mireille Dardenne and Jean-Marie Pleau, et al, “Contribution of zinc and other metals to the biological activity of the serum thymic factor,” Proceedings of the National Academy of Sciences, USA, September 1982, Vol. 79(19):5370-3.
M. Dardenne, W. Savino, S. Berrih, and J. F. Bach, “A zinc-dependent epitope on the molecule of thymulin, a thymic hormone”, Proceedings of the National Academy of Sciences, USA, October 1985, Vol. 82, pp. 7035-7038, https://www.ncbi.nlm.nih.gov/pmc/articles/PMC391304/pdf/pnas00360-0321.pdf
Jean-François Bach and Mireille Dardenne, “Thymulin, a zinc-dependent hormone,” Medical Oncology and Tumor Pharmacotherapy, March 1989, Volume 6, Issue 1, pp 25-29, https://link.springer.com/article/10.1007/BF02985220
Mireille Dardenne and Jean-Marie Pleau, “Interactions between Zinc and Thymulin,” Metal Based Drugs, 1994; 1(2-3): 233-239, https://www.ncbi.nlm.nih.gov/pmc/articles/PMC2364880/
Eugenio Mocchegiani, Paolo Paolucci, Donatella Granchi, Laura Cavallazzi, Lory Santarelli, and Nicola Fabris, “Plasma Zinc Level and Thymic Hormone Activity in Young Cancer Patients,” The American Society of Hematology, 1994, http://www.bloodjournal.org/content/bloodjournal/83/3/749.full.pdf?sso-checked=true
J. J. Haddad, N. E. Saadé and B. Safieh-Garabediana, “Thymulin: An Emerging Anti-Inflammatory Molecule,” Curr. Med. Chem. —Anti-Inflammatory & Anti Allergy Agents, 2005, 4, 333-338 333 1568-0142/05, http://citeseerx.ist.psu.edu/viewdoc/download?doi=10.1.1.658.4398&rep=rep1&type=pdf
Paula C. Reggiani, José I. Schwerd, Gloria M. Cónsole, Eduardo A. Roggero, Mireille Dardenne and Rodolfo G. Goya, “Physiology and Therapeutic Potential of the Thymic Peptide Thymulin,” Current Pharmaceutical Design, 2014, 20, 000-000 1 1381-6128/14. DOI: 10.2174/1381612820666140130211157

What is claimed is:

1. A pharmaceutical composition comprising thymulin, zinc, and one or more pharmaceutically acceptable excipients.

2. A pharmaceutical composition comprising thymulin, zinc, and one or more pharmaceutically acceptable excipients combined in vivo.

3. A pharmaceutical composition comprising thymulin, zinc, and one or more pharmaceutically acceptable excipients combined ex vivo.

4. The pharmaceutical composition according to claim 1, wherein the thymulin is sequenced and synthesized using the amino acid sequence Glu-Ala-Lys-Ser-Gln-Gly-Gly-SerAsn-OH.

5. The pharmaceutical composition according to claim 1, wherein the thymulin is sequenced and synthesized using the amino acid sequence H-Pyr-Glu-Ala-Lys-Ser-Gln-Gly-Gly-SerAsn-OH.

6. The pharmaceutical composition according to claim 1, wherein the thymulin is sequenced and synthesized using the amino acid sequence Pyro-Glu-Ala-Lys-Ser-GIn-GlyGly-Ser-Asn.

7. The pharmaceutical composition according to claim 1, wherein the thymulin is sequenced and synthesized using the amino acid sequence H-Pyr-Ala-Lys-Ser-Gln-Gly-Gly-Ser-Asn-OH.

8. The pharmaceutical composition according to claim 1, wherein the thymulin is sequenced and synthesized using the amino acid sequence Pyr-Ala-Lys-Ser-GIn-Gly-Gly-Ser-Asn-OH.

9. The pharmaceutical composition according to claim 1, wherein thymulin produced by thymic epithelial cells is biologically extracted from human plasma.

10. The pharmaceutical composition according to claim 1, wherein the thymulin is biologically extracted from porcine serum.

11. The pharmaceutical composition according to claim 1, wherein the thymulin is biologically extracted from bovine serum.

12. The pharmaceutical composition according to claim 1, wherein the thymulin is a synthetic peptide analog of thymulin.

13. The pharmaceutical composition according to claim 1, wherein the zinc is at least one selected from the group: zinc acetate, zinc chloride, zinc sulfate, zinc monomethionine, zinc picolinate, zinc gluconate, zinc aspartate, zinc citrate, zinc orotate, zinc glycinate, zinc oxide, and mixtures thereof.

14. The pharmaceutical composition according to claim 13, wherein the zinc is zinc chloride.

15. The pharmaceutical composition according to claim 1 wherein the zinc is present in an amount ranging from about 10 mg to about 200 mg.

16. The pharmaceutical composition according to any one of claim 1, wherein the zinc is present in an amount ranging from about 15 to 100 micromolar zinc.

17. The pharmaceutical composition according to claim 15, wherein the zinc is present in an amount of about 30 mg.

18. The pharmaceutical composition according to claim 15 wherein the zinc is present in an amount of about 50 mg.

19. The pharmaceutical composition according to claim 16 wherein the zinc is present in an amount of about 15 micromolar zinc.

20. The pharmaceutical composition according to claim 16 wherein the zinc is present in an amount of about 20 micromolar zinc.

21. The pharmaceutical composition according to claim 16 wherein the zinc is present in an amount of about 30 micromolar zinc.

22. The pharmaceutical composition according to claim 16 wherein the zinc is present in an amount of about 45 micromolar zinc.

23. A pharmaceutical composition according to claim 1 further comprising:
about 10 mg to about 200 mg zinc;
thymulin sequenced and synthesized using the amino acid sequence Glu-Ala-Lys-Ser-Gln-Gly-Gly-SerAsn-OH; and,
pharmaceutically acceptable excipients.

24. A pharmaceutical composition according to claim 1 further comprising:
about 10 mg to about 200 mg zinc;
thymulin sequenced and synthesized using the amino acid sequence H-Pyr-Glu-Ala-Lys-Ser-Gln-Gly-Gly-SerAsn-OH; and,
pharmaceutically acceptable excipients.

25. A pharmaceutical composition according to claim 1 further comprising:
about 10 mg to about 200 mg zinc;
thymulin sequenced and synthesized using the amino acid sequence Pyro-Glu-Ala-Lys-Ser-GIn-GlyGly-Ser-Asn; and,
pharmaceutically acceptable excipients.

26. A pharmaceutical composition according to claim 1 further comprising:
about 10 mg to about 200 mg zinc;
thymulin sequenced and synthesized using the amino acid sequence H-Pyr-Ala-Lys-Ser-Gln-Gly-Gly-Ser-Asn-OH; and,
pharmaceutically acceptable excipients.

27. A pharmaceutical composition according to claim 1 further comprising:
about 10 mg to about 200 mg zinc;
thymulin sequenced and synthesized using the amino acid sequence Pyr-Ala-Lys-Ser-GIn-Gly-Gly-Ser-Asn-OH; and,
pharmaceutically acceptable excipients.

28. A pharmaceutical composition according to claim 1 further comprising:
about 10 mg to about 200 mg zinc;
a synthetic peptide analog of thymulin; and,
pharmaceutically acceptable excipients.

29. A pharmaceutical composition according to claim 1 further comprising:
about 15 to 45 micromolar zinc;
thymulin sequenced and synthesized using the amino acid sequence Glu-Ala-Lys-Ser-Gln-Gly-Gly-SerAsn-OH; and,
pharmaceutically acceptable excipients.

30. A pharmaceutical composition according to claim 1 further comprising:
about 15 to 45 micromolar zinc;
thymulin sequenced and synthesized using the amino acid sequence H-Pyr-Glu-Ala-Lys-Ser-Gln-Gly-Gly-SerAsn-OH; and,
pharmaceutically acceptable excipients.

31. A pharmaceutical composition according to claim 1 further comprising:
about 15 to 45 micromolar zinc;
thymulin sequenced and synthesized using the amino acid sequence Pyro-Glu-Ala-Lys-Ser-GIn-GlyGly-Ser-Asn; and,
pharmaceutically acceptable excipients.

32. A pharmaceutical composition according to claim 1 further comprising:
about 15 to 45 micromolar zinc;
thymulin sequenced and synthesized using the amino acid sequence H-Pyr-Ala-Lys-Ser-Gln-Gly-Gly-Ser-Asn-OH; and,
pharmaceutically acceptable excipients.

33. A pharmaceutical composition according to claim 1 further comprising:
about 15 to 45 micromolar zinc;
thymulin sequenced and synthesized using the amino acid sequence Pyr-Ala-Lys-Ser-GIn-Gly-Gly-Ser-Asn-OH; and,
pharmaceutically acceptable excipients.

34. A pharmaceutical composition according to claim 1 further comprising:
about 15 to 45 micromolar zinc;
biologically extracted human thymulin; and,
pharmaceutically acceptable excipients.

35. A pharmaceutical composition according to claim 1 further comprising:
about 15 to 45 micromolar zinc;
biologically extracted porcine thymulin; and,
pharmaceutically acceptable excipients.

36. A pharmaceutical composition according to claim 1 further comprising:
about 15 to 45 micromolar zinc;
biologically extracted bovine thymulin; and,
pharmaceutically acceptable excipients.

37. A pharmaceutical composition according to claim 1 further comprising:
an equimolar concentration of zinc and thymulin; and,
pharmaceutically acceptable excipients.

38. A pharmaceutical composition according to claim 1 further comprising:
an equimolar concentration of zinc and synthetic thymulin; and,
pharmaceutically acceptable excipients.

39. A pharmaceutical composition according to claim 1 further comprising:
an equimolar concentration of zinc and natural thymulin; and,
pharmaceutically acceptable excipients.

40. A pharmaceutical composition according to claim 1 further comprising:
about 15 to 45 micromolar zinc;
an equimolar concentration of thymulin sequenced and synthesized using the amino acid sequence Glu-Ala-Lys-Ser-Gln-Gly-Gly-SerAsn-OH; and,
pharmaceutically acceptable excipients.

41. A pharmaceutical composition according to claim 1 further comprising:
about 15 to 45 micromolar zinc;
an equimolar concentration of thymulin sequenced and synthesized using the amino acid sequence H-Pyr-Glu-Ala-Lys-Ser-Gln-Gly-Gly-SerAsn-OH; and,
pharmaceutically acceptable excipients.

42. A pharmaceutical composition according to claim 1 further comprising:
about 15 to 45 micromolar zinc;
an equimolar concentration of thymulin sequenced and synthesized using the amino acid sequence Pyro-Glu-Ala-Lys-Ser-GIn-GlyGly-Ser-Asn; and,
pharmaceutically acceptable excipients.

43. A pharmaceutical composition according to claim 1 further comprising:
about 15 to 45 micromolar zinc;
an equimolar concentration of thymulin sequenced and synthesized using the amino acid sequence H-Pyr-Ala-Lys-Ser-Gln-Gly-Gly-Ser-Asn-OH; and,
pharmaceutically acceptable excipients.

44. A pharmaceutical composition according to claim 1 further comprising:
about 15 to 45 micromolar zinc;
an equimolar concentration of thymulin sequenced and synthesized using the amino acid sequence Pyr-Ala-Lys-Ser-GIn-Gly-Gly-Ser-Asn-OH; and,
pharmaceutically acceptable excipients.

45. A pharmaceutical composition according to claim 1 further comprising:
about 15 to 45 micromolar zinc;
an equimolar concentration of biologically extracted human thymulin; and,
pharmaceutically acceptable excipients.

46. A pharmaceutical composition according to claim 1 further comprising:
an equimolecular ratio of zinc and thymulin; and,
pharmaceutically acceptable excipients.

47. The pharmaceutical composition of claim 15, wherein the zinc is zinc chloride.

48. The pharmaceutical composition of claim 15, wherein a thymulin solution prepared from serum thymic factor is administered to subjects in an amount ranging from 10 picogram per milliliter to 4000 picogram per milliliter.

49. The pharmaceutical composition of claim 15, wherein a thymulin solution prepared from serum thymic factor is administered to subjects in the amount of 0.4 mg/kg body mass.

50. The pharmaceutical composition of claim 15, wherein a thymulin solution prepared from serum thymic factor is administered to subjects in the range of 0.1-100 μg/kg.

51. The pharmaceutical composition of claim 15, wherein a thymulin solution prepared from serum thymic factor is administered to subjects in the amount within the range of about 5 mg to 20 mg inclusive.

52. The pharmaceutical composition of claim 15, wherein a thymulin solution prepared from serum thymic factor is administered to subjects in the amount of 1.5 mg/kg body weight of the subject.

53. A pharmaceutical composition according to claim 1 further comprising: zinc and thymulin present at a weight/volume percentage of 0.0001%-0.1%.

54. A pharmaceutical composition according to claim 1 further comprising: zinc and thymulin present at a weight/volume percentage of 0.001%-0.05%.

55. A pharmaceutical composition according to claim 1 further comprising:
about 10 mg to about 200 mg zinc;
about 5 to 20 mg of thymulin; and,
pharmaceutically acceptable excipients.

56. A pharmaceutical composition of claim 1 further comprising:
about 15 micromolar to 100 micromolar zinc;
about 0.1 mg/kg to 10 mg/kg body mass thymulin; and,
pharmaceutically acceptable excipients.

57. A pharmaceutical composition of claim 1 further comprising:
about 30 mg zinc;
about 0.4 mg/kg of body mass of thymulin; and,
pharmaceutically acceptable excipients.

58. A pharmaceutical composition of claim 1 further comprising:
about 15 micromolar zinc;
about 0.4 mg/kg of body mass of thymulin; and,
pharmaceutically acceptable excipients.

59. A pharmaceutical composition comprising:
about 15 to 20 micromolar zinc; and,
pharmaceutically acceptable excipients.

60. A pharmaceutical composition comprising:
the recombinant adenoviral vector, RAd-metFTS (methionine-FTS), expressing the synthetic DNA sequence encoding met-FTS;
an analog of Serum Thymic Factor (FTS); and,
pharmaceutically acceptable excipients.

61. The pharmaceutical composition according to claim 1 wherein a solid oral dosage is a capsule or tablet.

62. The pharmaceutical composition according claim 1, wherein the pharmaceutical composition is formulated as a powder.

63. The pharmaceutical composition according to claim 1, wherein the pharmaceutical composition is formulated as at least one selected from the group of: a liquid, a suspension, injectable solution, and syrup.

64. The pharmaceutical composition according to claim 1, wherein the pharmaceutical composition is formulated and administered parenterally through subcutaneous administration.

65. The pharmaceutical composition according to claim 1, wherein the pharmaceutical composition is formulated and administered parenterally through intravenous administration.

66. The pharmaceutical composition according to claim 1, wherein the pharmaceutical composition is formulated and administered parenterally through intramuscular administration.

67. The pharmaceutical composition according to claim 1, wherein the pharmaceutical composition is formulated and administered through transdermal administration.

68. The pharmaceutical composition according to claim 1, wherein the pharmaceutical composition is formulated and administered in the form of a suppository.

69. A method for measuring and optimizing active thymulin in the pharmaceutical composition of claim 1 comprising:
providing thymulin;
providing zinc;
providing at least one pharmaceutically acceptable excipient;
combining the thymulin, zinc and pharmaceutically acceptable excipient into a pharmaceutical composition containing activated thymulin;
administering the pharmaceutical composition to a subject;
withdrawing at least one serum sample from the subject; and,
measuring at least one selected from the group of: thymulin, zinc, and the pharmaceutically acceptable excipient.

70. The method of claim 69 wherein the presence of active thymulin is monitored and optimized by detecting the zinc present in thymulin at a subatomic level using X-Ray fluorescence.

71. The method of claim 70 wherein zinc is added to the composition until it shows the presence of active thymulin. through the method of claim 72.

72. The method of claim 69 wherein zinc is mixed with thymulin in an equimolar concentration.

73. The method of claim 69 wherein zinc is mixed with thymulin in an equimolecular ratio.

74. The method of claim 69 wherein thymulin is measured using antibodies.

75. The method of claim 69 wherein the presence of zinc in the thymulin is confirmed using X Ray fluorescence techniques once it has been added to the thymulin.

76. The method of claim 69 wherein the measurement method is an enzyme-linked immunosorbent assay (ELISA) kit that determines the presence of thymulin in the composition within a range of 0.03-16 ng mL⁻¹.

77. The method of claim 69 wherein the measurement method is a radioimmunoassay of the composition using an antibody specific for thymulin.

78. The method of claim 69 wherein the measurement method is the measurement of zinc is accomplished via atomic absorption spectroscopy.

79. A method for generating T Helper 1 cells which produce Interleukin 2 which then activates cytotoxic T cells comprising: administering, to a subject in need thereof, a pharmaceutical composition according to claim 1.

80. A method for treating, reducing the severity of, reducing the incidence of, delaying the onset of, preventing a relapse to or reducing pathogenesis of a chronic condition associated with an immune system dysfunction or deficiency comprising administering, to a subject in need thereof, a pharmaceutical composition according to claim 1.

81. The method according to claim 80, wherein the chronic condition is cancer.

82. The method according to claim 81, wherein the cancer is at least one selected from the group of: pancreatic cancer, prostate cancer, breast cancer, lung cancer, colon cancer, cervical cancer, ovarian cancer, melanoma, lymphoma, and squamous cell carcinoma.

83. A method for treating, reducing the severity of, reducing the incidence of, delaying the onset of, preventing a relapse to or reducing pathogenesis of a persistent infection comprising: administering, to a subject in need thereof, a pharmaceutical composition according to claim 1.

84. The method according to claim 83, wherein the persistent infection is at least one selected from the group of: a viral infection, a bacterial infection, a fungal infection, and a parasitic infection.

85. The method according to claim 84, wherein the viral infection is an infection with at least one selected from the group of: a hepatitis virus, a human immunodeficiency virus (HIV), a human T-lymphotrophic virus (HTLV), a herpes virus, an Epstein-Barr virus, and a human papilloma virus.

86. The method according to claim 79 further comprising: wherein cytotoxic T-cell activity is increased by administering, to a subject in need thereof, the pharmaceutical composition according to claim 1.

87. The method according to claim 86, wherein the cytotoxic T-cell activity is at least one selected from the group of: cytokine production, T cell proliferation, and infectious agent clearance.

88. The method according to claim 87, wherein the infectious agent is at least one selected from the group of: a virus, a bacterium, a fungus, a mycoplasm, and a parasite.

89. The method according to claim 87, wherein the T-cell is a cancer/tumor antigen-specific T cell.

90. The method according to claim 87, wherein the cytokine is at least one selected from the group of: IFNγ, TNFα, or IL-2.

91. A method for diagnosing a subject as having or being at risk of having a persistent infection or cancer comprising:
(a) securing a plasma sample from the subject;
(b) measuring the biological activity of thymulin in the sample;
wherein a decrease in the biological activity of the thymulin compared to such activity in a control sample identifies the subject as having or at risk of having a persistent infection or cancer.

92. A method for selecting a treatment for a subject having or being at risk of having a persistent infection or cancer comprising the steps of:
(a) securing a plasma sample from the subject;
(b) measuring the biological activity of thymulin in the sample; and,
(c) selecting a treatment for the subject diagnosed as having or being at risk of a persistent infection or cancer comprising administering, to the subject in need thereof, a pharmaceutical composition according to claim 1.

93. The method according to claim 69, wherein the pharmaceutical composition is administered multiple times per day.

94. The method according to claim 69, wherein the pharmaceutical composition is administered one time per day.

95. The method according to claim 69, wherein the pharmaceutical composition is administered every second day or every third day.

96. A pharmaceutical composition comprising:
thymulin;
zinc; and,
one or more pharmaceutically acceptable excipients;
wherein the thymulin and zinc are combined in solution each at a 15 μmolar concentration along with the one or more pharmaceutically acceptable excipients; the resulting composition suitable for parenteral administration to a subject.


SEQUENCE LISTING (1 sequence)

SEQ ID NO: 1 - protein, 9 aa, Artificial Sequence
  Product of the Human Mind
EAKSQGGSN